(19) United States
(12) Patent Application Publication
(10) Pub. No.: US 2012/0197071 A1
(43) Pub. Date: Aug. 2, 2012

(54) FINGER CONDOM

(76) Inventor: Han-Huei Liu, Taipei City (TW)

(21) Appl. No.: 13/019,679

(22) Filed: Feb. 2, 2011

(51) Int. Cl. A61F 5/00; A41D 13/08

(52) U.S. Cl. 600/38; 2/21

(57) ABSTRACT

A finger condom having an elastic hollow tubular sheath having an opening at a bottom end thereof, a closed end portion at a top end thereof and a tubular body portion connected between the opening and the closed end portion and a non-uniform diameter with the largest diameter at the bottom end and the smallest diameter at the top end, and an elastic band formed integral with the bottom end of the elastic hollow tubular sheath around the opening.

### BACKGROUND OF THE INVENTION

1. Field of the Invention

The present invention relates to finger condoms and more particularly, to such a non-uniform diameter type finger condom, which a finger condom, which facilitates application and even distribution of lubricant.

2. Description of the Related Art

To reduce the probability of pregnancy and spreading sexually transmitted diseases (STDs—such as gonorrhea, syphilis, and HIV) a condom may be used during sexual intercourse. Many condoms have been disclosed. Exemplars are seen in US 2005-0076917 entitled “Lubricated Condom”; U.S. Pat. No. 5,137,032 entitled “Natural Feeling Flexible Contraceptive Device”; U.S. Pat. No. 5,163,448 entitled “Condom Comprising Dispensing Structure, and Method of Making and Using the Same”; U.S. Pat. No. 5,351,698 entitled “Bidirectionally Donnable Generally Tubular Sheath Articles, and Apparatus and Method for making and Using Same”. Many female contraceptive devices are also known, such as US 2006-0231104 entitled “Female Barrier Contraceptive with Vacuum Anchoring” The aforesaid various conventional condom designs are to be put on the external genital organ and commonly have a uniform diameter, not suitable for use with the finger.

US 2005-0076917 and US 2006-0231104 both designs indicate finger applicability. However, due to structural design limitations, they cannot be firmly and comfortably put on the finger.

Following openness of sexual concept, the concept of finger sex by using a finger to insert into the vagina or anus instead of conventional sexual intercourse is acceptable to most people, and can bring plenty of happiness. More particularly at the present time of rising awareness of women's rights, finger sex can bring a woman to an orgasm easily. However, when playing a finger sex, it is easy to spread diseases.

To reduce the probability of spreading diseases during a finger sex activity, the inventor of the present invention invented a finger condom, as indicated in US2010-0037361. According to this design, the finger condom includes a flexible finger condom body that has an open end, a closed end and lubricated inner and outer surfaces and defines a volume smaller than one finger of a person before use, and an elastic band extending around the periphery.

When the finger condom is put on the finger, the finger is well enclosed, assuring application safety and reducing the probability of spreading diseases. However, because the flexible finger condom body has a uniform diameter, it does not fit the configuration of a human finger perfectly, causing discomfort in the proximal end of the finger that is wrapped by the elastic band, and the applied lubricant may be not evenly distributed. An improvement in this regard is necessary.

### SUMMARY OF THE INVENTION

The present invention has been accomplished under the circumstances in view. It is one object of the present invention to provide a finger condom, which facilitates application and even distribution of lubricant.

To achieve this and other objects of the present invention, a finger condom comprises an elastic hollow tubular sheath having an opening at a bottom end thereof, a closed end portion at a top end thereof and a tubular body portion connected between the opening and the closed end portion and having a non-uniform diameter with the smallest diameter at the top end thereof and the largest diameter at the bottom end thereof, and an elastic band formed integral with the bottom end of the tubular body portion around the opening.

As the finger condom has the largest diameter at the opening at the bottom end of the tubular body portion, the user can sleeve the tubular body portion onto the finger rapidly and comfortably. When the tubular body portion is fully attached to the finger, it is firmly secured in position. Therefore, the invention facilitates application and even distribution of lubricant.

Further, the tubular body portion has the smallest diameter thereof ranging from 29 mm to 32 mm, and the largest diameter thereof ranging from 24 mm to 36 mm.

Further, the tubular body portion can be made having the diameter thereof gradually increasing from the top end to the bottom end. Alternatively, the tubular body portion can be made having the smallest diameter thereof extending downwardly from the top end thereof to ⅓ of the length thereof and then gradually increasing to the largest diameter at the bottom end thereof.

### BRIEF DESCRIPTION OF THE DRAWINGS

FIG. 1 is an elevational view of a finger condom in accordance with a first embodiment of the present invention.

FIG. 2 is a sectional view of the finger condom in accordance with the first embodiment of the present invention.

FIG. 3 is a schematic drawing illustrating an application example of the finger condom in accordance with the first embodiment of the present invention.

FIG. 4 is a perspective view of a finger condom in accordance with a second embodiment of the present invention.

### DETAILED DESCRIPTION OF THE PREFERRED EMBODIMENT

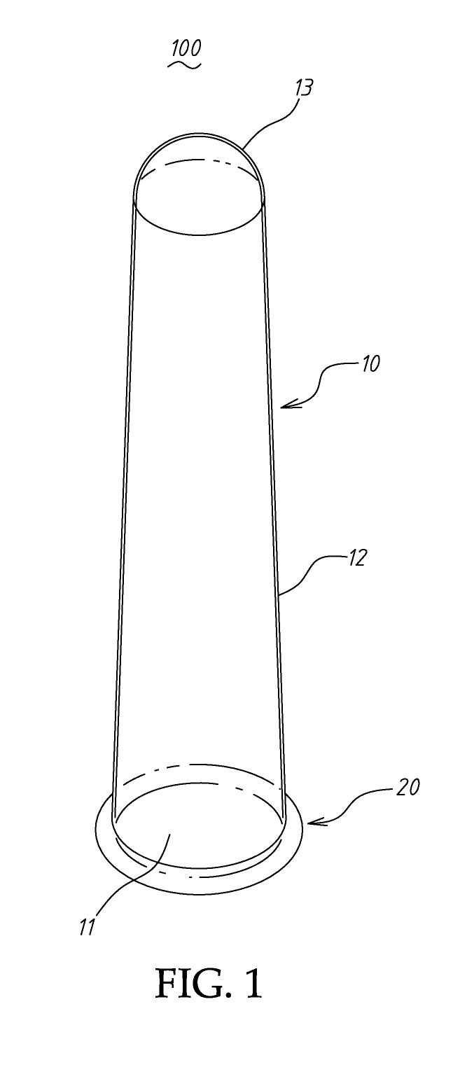

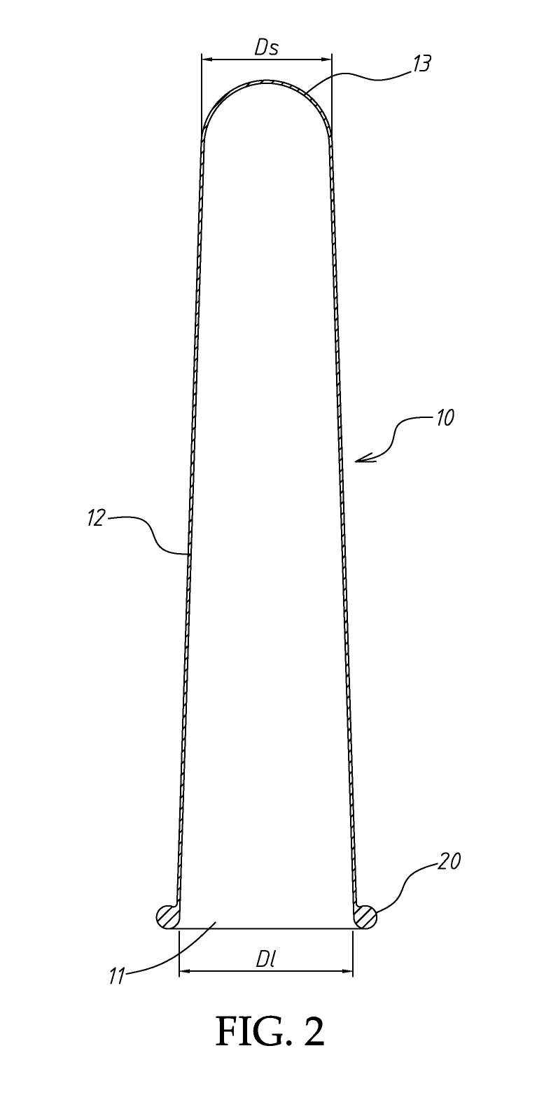

Referring to FIGS. 1 and 2, a finger condom 100 in accordance with a first embodiment of the present invention is shown comprising an elastic hollow tubular sheath 10 and an elastic band 20.

The elastic hollow tubular sheath 10 has an opening 11 at a proximal end thereof, a closed end portion 13 at a distal end thereof and a tubular body portion 12 connected between the opening 11 and the closed end portion 13. The closed end portion 13 is rounded and connected to one end, namely, the top end, of the tubular body portion 12. The opening 11 is located on the other end, namely, the bottom end, of the tubular body portion 12 for the insertion of a finger into the tubular body portion 12. The elastic band 20 is arranged around the opening 11 at the bottom end of the tubular body portion 12. Further, the elastic band 20 is formed integral with the bottom end of the elastic hollow tubular sheath 10.

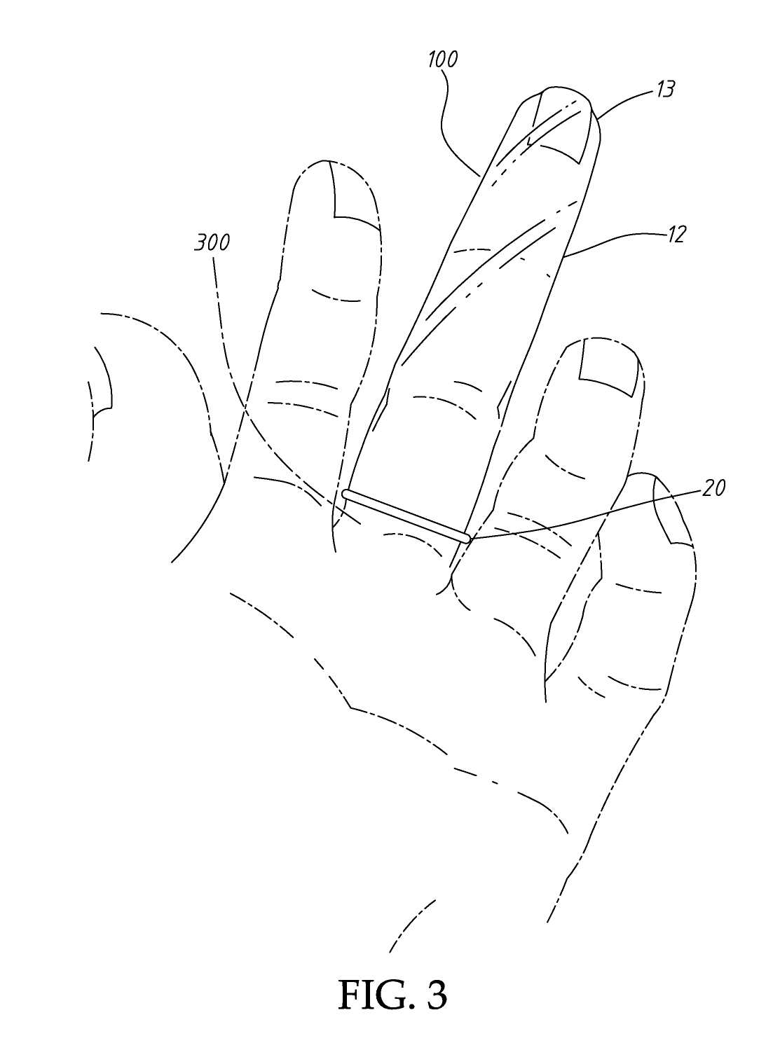

The main feature of the design of the resent invention is that the tubular body portion 12 has a non-uniform diameter. As shown in FIG. 2, the top end of the tubular body portion 12 has the smallest diameter Ds ranging from 20 to 32 mm, or preferably, 24 mm; the bottom end of the tubular body portion 12 has the largest diameter D1 ranging from 24 to 36 mm, or preferably, 28 mm. Subject to this non-uniform diameter design, the finger condom 100 fits the configuration of a human finger, and can be secured to one finger 300 of a person firmly, as shown in FIG. 3.

According to the first embodiment of the present invention, as shown in FIGS. 1 and 2, the tubular body portion 12 has a diameter increasing gradually from the smallest diameter Ds at the top end of the tubular body portion 12 toward the largest diameter D1 at the bottom end of the tubular body portion 12.

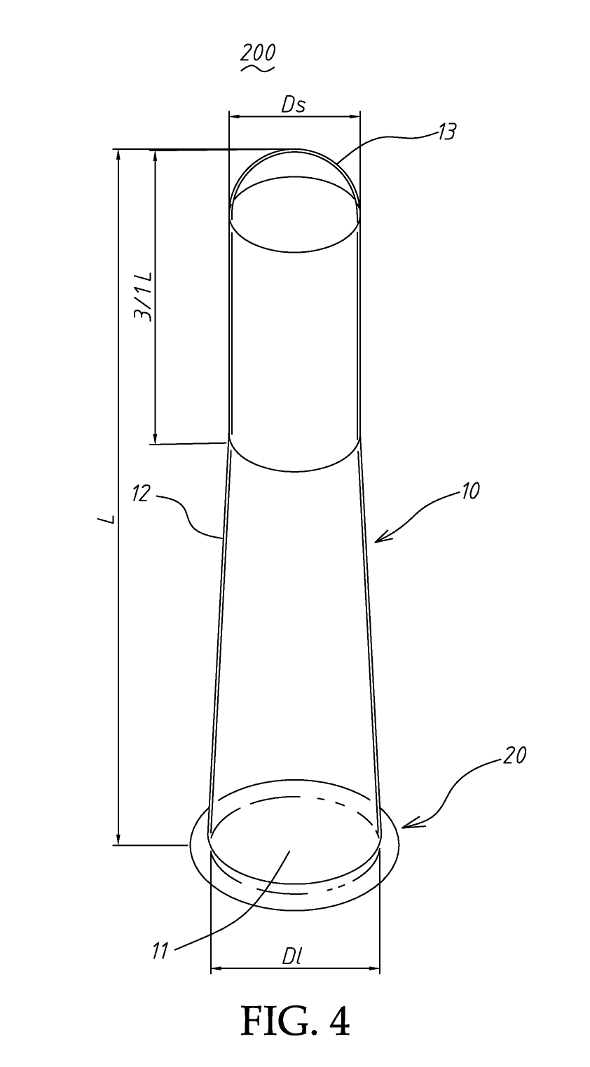

FIG. 4 illustrates a finger condom 200 in accordance with a second embodiment of the present invention. Similar to the aforesaid first embodiment, the finger condom 200 an elastic hollow tubular sheath 10 and an elastic band 20. The elastic hollow tubular sheath 10 has an opening 11 at a proximal end thereof, a closed end portion 13 at a distal end thereof and a tubular body portion 12 connected between the opening 11 and the closed end portion 13. However, according to this second embodiment, the diameter of the tubular body portion 12 has the smallest diameter Ds extending downwardly from the top end of the tubular body portion 12 toward the bottom end of the tubular body portion 12 to about ⅓ of the length L of the tubular body portion 12, and then gradually increasing to the largest diameter D1 at the bottom end of the tubular body portion 12. Subject to this non-uniform diameter design, the finger condom 200 fits the configuration of a human finger, and can be secured to one finger 300 of a person firmly.

According to the present invention, the finger condom has the largest diameter D1 at the opening 11 at the bottom end of the tubular body portion 12. Thus, the user can sleeve the tubular body portion 12 onto the finger 300 rapidly and comfortably. When the tubular body portion 12 is fully attached to the finger 300, it is firmly secured in position. When compared to conventional finger condoms with a uniform diameter, the invention facilitates application and even distribution of lubricant.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A finger condom, comprising:
an elastic hollow tubular sheath having an opening at a bottom end thereof, a closed end portion at a top end thereof and a tubular body portion connected between said opening and said closed end portion, said closed end portion being rounded, said tubular body portion having a non-uniform diameter, the smallest diameter at the top end thereof and the largest diameter at the bottom end thereof; and
an elastic band formed integral with the bottom end of said tubular body portion around said opening.

2. The finger condom as claimed in claim 1, wherein said tubular body portion has the smallest diameter thereof ranging from 29 mm to 32 mm, and the largest diameter thereof ranging from 24 mm to 36 mm.

3. The finger condom as claimed in claim 1, wherein said tubular body portion has the diameter thereof gradually increasing from the smallest diameter at the top end thereof to the largest diameter at the bottom end thereof.

4. The finger condom as claimed in claim 1, wherein said tubular body portion has the smallest diameter at the top end thereof toward the largest diameter at the bottom end thereof to ⅓ of the length of said tubular body portion and then gradually increasing to the largest diameter at the bottom end thereof.

5. A finger condom, comprising:
an elastic hollow tubular sheath having an opening at a bottom end thereof, a closed end portion at a top end thereof and a tubular body portion connected between said opening and said closed end portion, said closed end portion being rounded, said tubular body portion having a non-uniform diameter gradually increasing from the smallest diameter at the top end thereof to to the largest diameter at the bottom end thereof; and
an elastic band formed integral with the bottom end of said tubular body portion around said opening.

6. The finger condom as claimed in claim 5, wherein said tubular body portion has the smallest diameter thereof ranging from 29 mm to 32 mm, and the largest diameter thereof ranging from 24 mm to 36 mm.

7. A finger condom, comprising:
an elastic hollow tubular sheath having an opening at a bottom end thereof, a closed end portion at a top end thereof and a tubular body portion connected between said opening and said closed end portion, said closed end portion being rounded, said tubular body portion having a non-uniform diameter, the smallest diameter extending downwardly from the top end thereof to ⅓ of the length of said tubular body portion and then gradually increasing to the largest diameter at the bottom end thereof; and
an elastic band formed integral with the bottom end of said tubular body portion around said opening.

8. The finger condom as claimed in claim 7, wherein said tubular body portion has the smallest diameter thereof ranging from 29 mm to 32 mm, and the largest diameter thereof ranging from 24 mm to 36 mm.
